(19) United States
(12) Patent Application Publication
(10) Pub. No.: US 2007/0055379 A1
(43) Pub. Date: Mar. 8, 2007

(54) ANNULAR ACCESS DEVICES

(76) Inventors: Corbett W. Stone, San Diego, CA (US); Jennifer Diederich, Cumberland, RI (US); Bryan D. Knodel, Flagstaff, AZ (US)

Correspondence Address: CARR LLP (IST), 670 FOUNDERS SQUARE, 900 JACKSON STREET, DALLAS, TX, 75202, US

(21) Appl. No.: 11/462,319

(22) Filed: Aug. 3, 2006

Related U.S. Application Data
(60) Provisional application No. 60/705,122, filed on Aug. 3, 2005.
(60) Provisional application No. 60/720,848, filed on Sep. 27, 2005.
(60) Provisional application No. 60/780,897, filed on Mar. 9, 2006.

(51) Int. Cl. A61F 2/44

(52) U.S. Cl. 623/17.16

(57) ABSTRACT

An annular access device provides minimally invasive access to the interior of an intervertebral disc, a stable working platform for treatment, and closure of an annulotomy. A method of performing a surgical operation on an intervertebral disc provides for inserting an obturator into an annulotomy, and actuating an operable member to secure the obturator to an annulus of an intervertebral disc.

### CROSS-REFERENCE TO RELATED APPLICATIONS

This application relates to, and claims the benefit of the filing date of co-pending U.S. provisional patent application Ser. No. 60/705,122 entitled “ANNULAR ACCESS DEVICES” filed Aug. 3, 2005, and co-pending U.S. provisional patent application Ser. No. 60/720,848 entitled “SYSTEM AND METHOD TO DELIVER ANCHORS INTO ANNULUS TISSUE” filed Sep. 27, 2005, and co-pending U.S. provisional patent application Ser. No. 60/780,897 entitled “ANNULAR ACCESS DEVICE USING T-ANCHORS” filed Mar. 9, 2006, the entire contents of which are incorporated herein by reference for all purposes.

### TECHNICAL FIELD

The invention relates generally to medical devices for treatment of spinal injuries and, more particularly, to devices for providing a means of gaining access to the annulus of an intervertebral disc.

### BACKGROUND

Minimally invasive access to the anterior spinal disc space for the purposes of tissue removal or treatment of the intervertebral disc space has been a difficult problem. As partial disc replacement (PDR) therapy becomes the standard of care, this access problem must be addressed by the suppliers of that treatment. One of the main obstacles PDR treatment techniques must overcome in order to become widely accepted is the extrusion of the nuclear replacement medium. Closure of the annulotomy after disc replacement is clearly desirable. Traditionally, suture closures are the most trusted techniques among surgeons. Suture closures are considered by the U.S. Food and Drug Administration to be relatively low risk compared to other implants. Fibrin glue closures have been evaluated for these kinds of closures, but are severely limited by their relatively low strength.

The standard of care for access to intervertebral space depends upon the procedure adopted. The mini-discectomy procedure has become popular for treatment of herniations, but this procedure is potentially more invasive than the procedures contemplated by the present invention and is sometimes used as a stop-gap treatment for patients who are considered likely to extrude a disc again. Fusion procedures still utilize the “fusion cage” and bone graft implants which require intrusion into the posterior spine elements and are frequently nearly as intrusive as traditional open fusion. The latest PDR treatments have a potential weakness in terms of extrusion of implanted material. What is needed is an adequate annulotomy closure means for eliminating the potential for extrusion of implanted material in PDR treatments.

### SUMMARY

An annular access device provides minimally invasive access to the interior of an intervertebral disc, a stable working platform for treatment, and closure of an annulotomy. A method of performing a surgical operation on an intervertebral disc provides for inserting an obturator into an annulotomy, and actuating an operable member to secure the obturator to an annulus of an intervertebral disc.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 is a perspective view of an embodiment of an annular access device;

FIG. 2 is a sectional view of an annular access device taken along the lines 2-2 in FIG. 1;

FIGS. 3A-B are detail sectional views of an annular access device taken within the areas 3A and 3B, respectively in FIG. 2;

FIG. 4 is a perspective view of an annular access device penetrating the annulus of an intervertebral disc;

FIGS. 5A-5D are perspective detail views, in partial cutaway, of an annular access device showing operation of needles and needle catchers;

FIG. 6 is a perspective view of an embodiment of an annular access device;

FIG. 7 is a sectional view of an embodiment of an annular access device taken along the lines 7-7 in FIG. 6;

FIGS. 8A-B are detail sectional views of an annular access device taken within the area 8 in FIG. 7;

FIG. 9 is a perspective view of a flexible anchor with flexible barbs;

FIG. 10A is a detail perspective view of flexible barbed anchors deployed from the tip of the annular access device shown in FIG. 6;

FIG. 10B is a detail sectional view a flexible barbed anchor deployed from the tip of the annular access device taken along the lines 10B-10B in FIG. 10A;

FIG. 11 is a perspective view of an embodiment of an annular access device;

FIG. 12 is a perspective view of an annular access device showing details of the actuation mechanism;

FIG. 13 is a detail perspective view of the handle of an annular access device showing details of the actuation mechanism;

FIGS. 14A-14C are perspective views on an annular access device showing actuation of the anchors;

FIGS. 15A-15C are detail perspective views of the handle of an annular access device showing operation of the actuation mechanism;

FIGS. 16A-16B are plan views of an embodiment of an annular access device;

FIGS. 17A-17B are perspective views of a staple device in un-deployed and deployed states;

FIG. 18 is a perspective view of an anvil subassembly used in the device shown in FIG. 16A-16B;

FIG. 19 is a perspective view of the distal end of an embodiment of an annular access device;

FIG. 20 is a cutaway view of the distal end of the annular access device shown in FIG. 19;

FIG. 21 is another cutaway view of the distal end of the annular access device shown in FIG. 19;

FIG. 22 is a cutaway view of the annular access device showing anchors deployed;

FIG. 23A is another cutaway view of the annular access device showing anchors deployed;

FIG. 23B is a schematic diagram of suture routing used with the annular access device;

FIG. 24 is perspective view of sutures routed through anchor pulleys;

FIG. 25 is a perspective view of an embodiment of a suture locking device;

FIG. 26 is a plan view of the suture locking device;

FIG. 27 is a sectional view of the suture locking device, taken along the lines 27-27 in FIG. 26;

FIG. 28 is a perspective view of a suture locking mechanism, viewed from a proximal end thereof; and

FIG. 29 is an exploded view of the suture locking mechanism, viewed from a distal end thereof.

### DETAILED DESCRIPTION

The entire contents of commonly assigned U.S. patent application Ser. No. 60/611,231, entitled “SYSTEM AND METHOD FOR MINIMALLY INVASIVE NUCLECTOMY,” filed Sep. 17, 2004, is hereby incorporated by reference for all purposes. The entire contents of co-pending U.S. provisional patent application Ser. No. 60/705,122 entitled “ANNULAR ACCESS DEVICES” filed Aug. 3, 2005, and co-pending U.S. provisional patent application Ser. No. 60/720,848 entitled “SYSTEM AND METHOD TO DELIVER ANCHORS INTO ANNULUS TISSUE” filed Sep. 27, 2005, and co-pending U.S. provisional patent application Ser. No. 60/780,897 entitled “SYSTEM AND METHOD TO ACCESS ANNULUS TISSUE USING T ANCHORS” filed Mar. 9, 2006, are hereby incorporated by reference for all purposes.

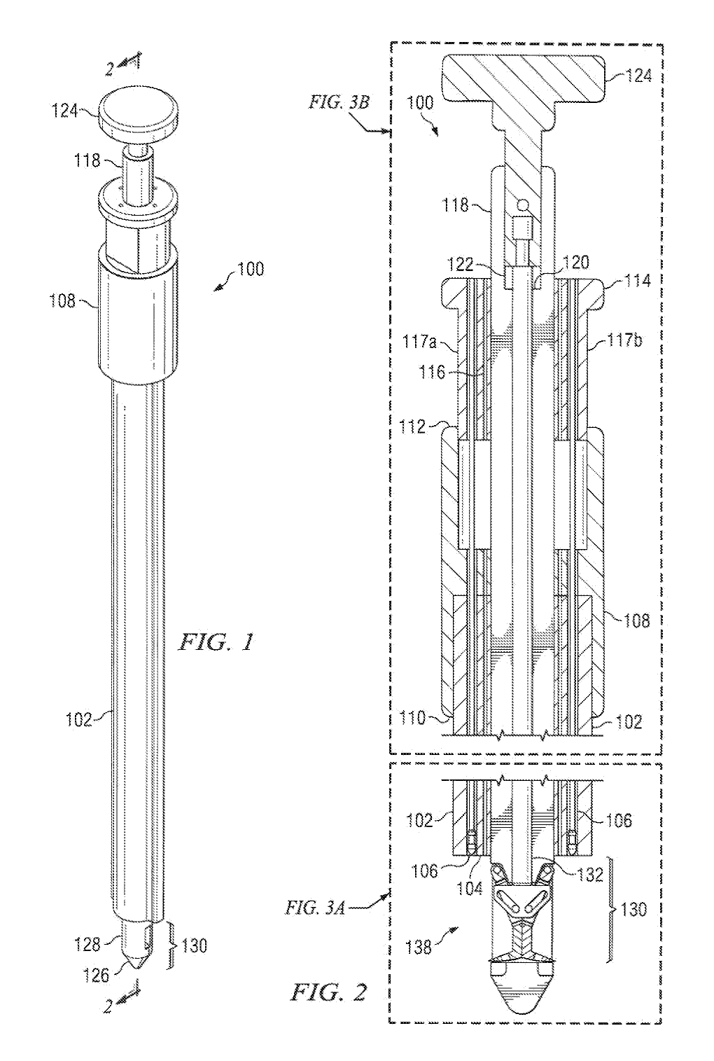

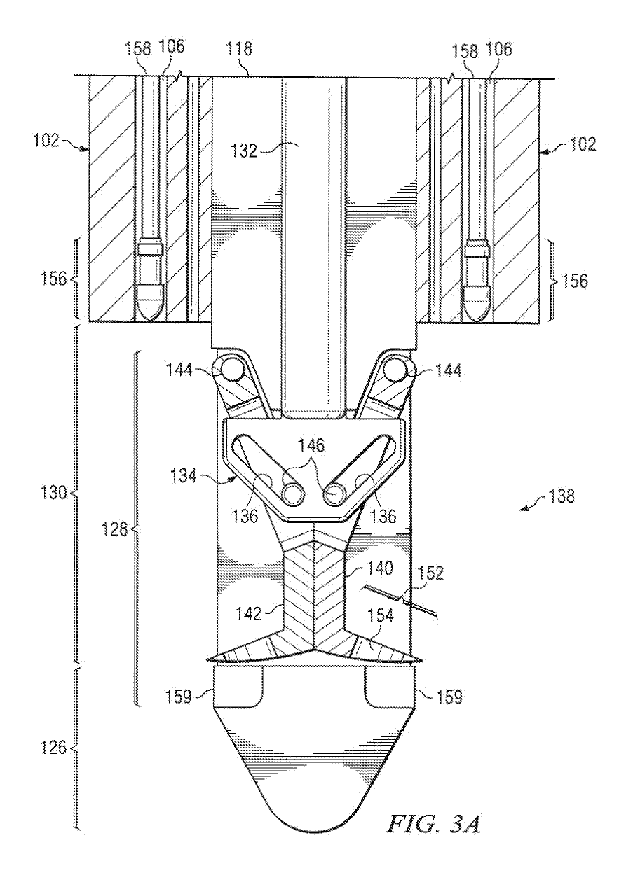

FIGS. 1 and 2 depict an embodiment of an annular access device 100 for providing minimally invasive access to the interior of an intervertebral disc, a stable working platform for treatment, and closure of the annulotomy. The annular access device 100 comprises an outer tube 102 having a central channel 104 running lengthwise therethrough and at least two needle channels 106 running lengthwise on opposite sides of the central channel 104. An outer tube cap 108, having a first cavity 110 at a distal end thereof and a second cavity 112 at a proximal end thereof, is coupled to a proximal end of the outer tube 102 whereby the proximal end of the outer tube 102 fits into the first cavity 110. An actuator handle 114 coupled to cap 108 at second cavity 112 is generally cylindrical, having a central channel 116 running lengthwise therethrough and two opposing side channels 117a and 117b. An inner tube 118 disposed within central channel 104 of outer tube 102 extends beyond the distal end of outer tube 102 and further extends through actuator handle 114 at the proximal end of outer tube 102.

The inner tube 118 is generally cylindrical and has a working channel 120 formed lengthwise through the center thereof. The diameter of the working channel 120 is generally constant through its length, but is greater at a proximal end to form a receptacle 122 for receiving a control knob 124. At a distal end of inner tube 118 is formed a generally conical tip 126 having a rounded point so that the inner tube 118 functions as an obturator. Behind the tip 126, the working channel 120 opens to a space forming deployment slots 128. The outer tube 102 then functions as a cannula and the inner tube 118 functions as an obturator having a deployment portion 130 formed at its end. A drive rod 132 slidably disposed within working channel 120 extends into receptacle 122 at the proximal end of inner tube 118 and is coupled to the control knob 124. Drive rod 132 is generally cylindrical and extends into the deployment portion 130 of inner tube 118.

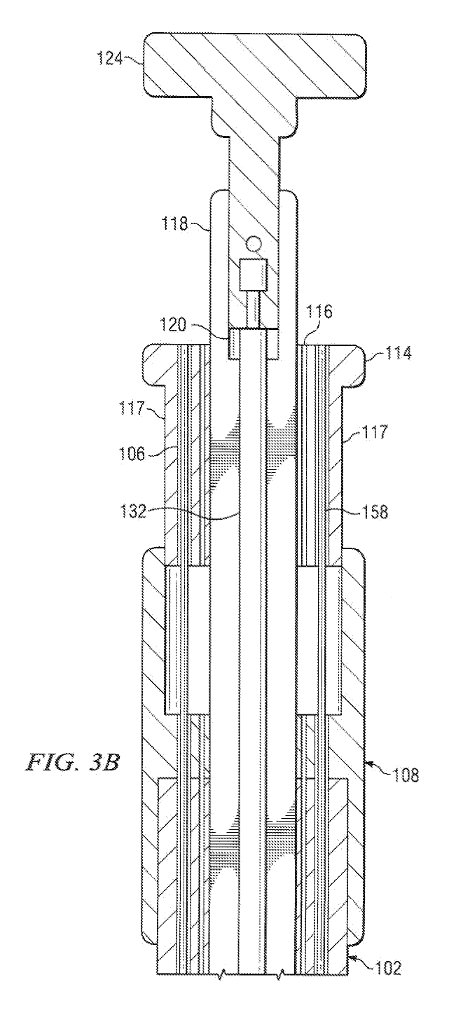

Referring now to FIG. 3A, which depicts a detail sectional representation of a needle catcher mechanism 138. An actuator plate 134 at the distal end of drive rod 132 has at least two drive slots 136 formed diagonally therein for coupling to the needle catcher mechanism 138 mounted in the deployment portion 130 of inner tube 118. A left-hand needle catcher 140 and a right-hand needle catcher 142 are mounted for pivotal movement within deployment slots 128 formed within the deployment portion 130 of inner tube 118. The needle catchers 140, 142 are pivotally secured at proximal ends thereof by pivot pins 144, and are coupled to actuator plate 134 by drive pins 146. The drive pins 146 pass through drive slots 136 in actuator plate 134 and are secured to the needle catchers 140, 142 at corresponding holes formed therein. Each needle catcher 140, 142 has a slot formed in a central portion thereof for allowing actuator plate 134 to fit within. Each needle catcher 140, 142 also has a hook portion 152 at a distal end thereof, and having an aperture 154 for capturing needle heads 156 as described hereinafter. Needle heads 156 are initially disposed at the distal ends of needle channels 106. Sutures (not shown) attached to the needle heads 156 lie within needle pusher tubes 158 disposed within needle channels 106 behind the needle heads 156. In operation, the needle catchers 140, 142 are deployed by pivoting outward to place hook portions 152 and apertures 154 in the path of needle heads 156. Recesses 159 formed at the back of obturator portion 126 adjacent deployment slots 128 receive the distal ends of needle heads 156 when needle catchers 140, 142 are retracted.

FIG. 3B depicts a detail sectional representation of actuator handle 114 and control knob 124 as coupled to drive rod 132. Control knob 124 and inner tube 118 are configured to be locked in one position to prevent actuation of the needle catcher mechanism 138. The surgeon can twist the control knob 124 to unlock the mechanism, and then push the control knob 124 to deploy the needle catcher mechanism 138. As control knob 124 is pushed, drive rod 132 slides through working channel 120 and extends actuator plate 134 to deploy the needle catchers 140, 142. The surgeon can then twist control knob 124 to lock the needle catcher mechanism 138 in the deployed position and press actuator handle 114 to extend the needle heads 156. Proximal ends of needle pusher tubes 158 fit within the opposing side channels 117 formed in actuator handle 114 and are extended as the handle 114 is pushed thereby extending the needle heads 156.

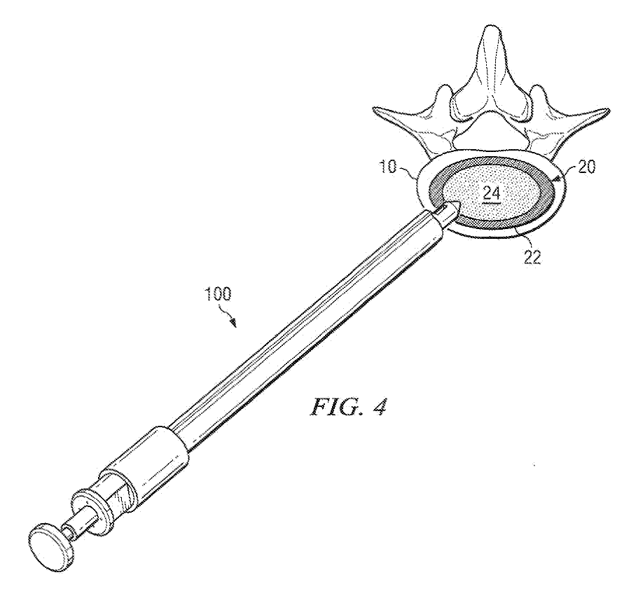

FIG. 4 depicts annular access device 100 used in contact with an intervertebral disc 20, comprising an annulus 22 and a nucleus 24, between adjacent vertebrae 10. Typically a trephine is used to cut an initial opening in the annulus 22. Thereafter, the blunt obturator portion and the cannula portion of the annular access device 100 are used to gain access to the nucleus 24.

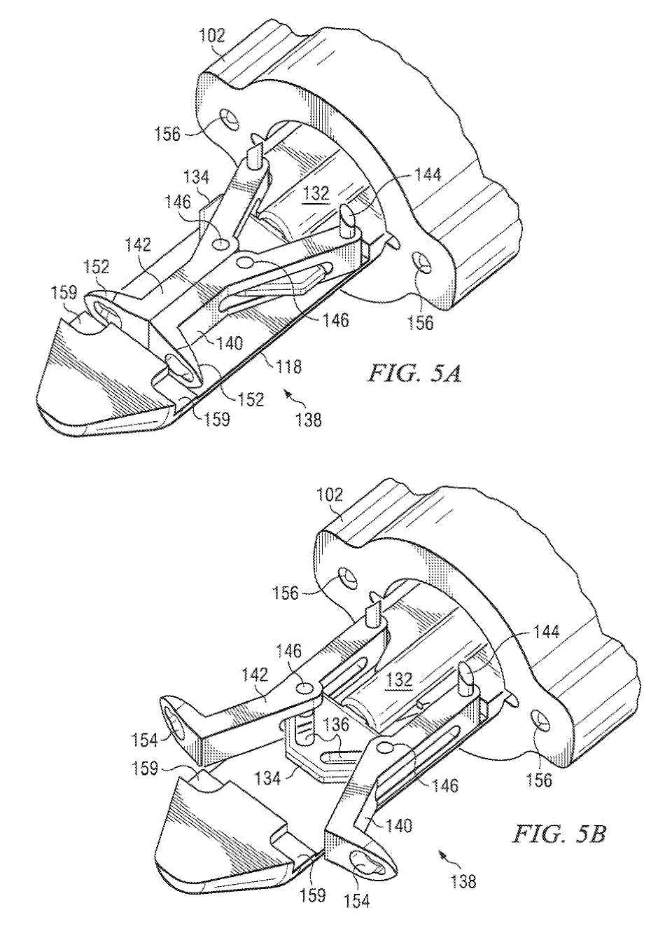

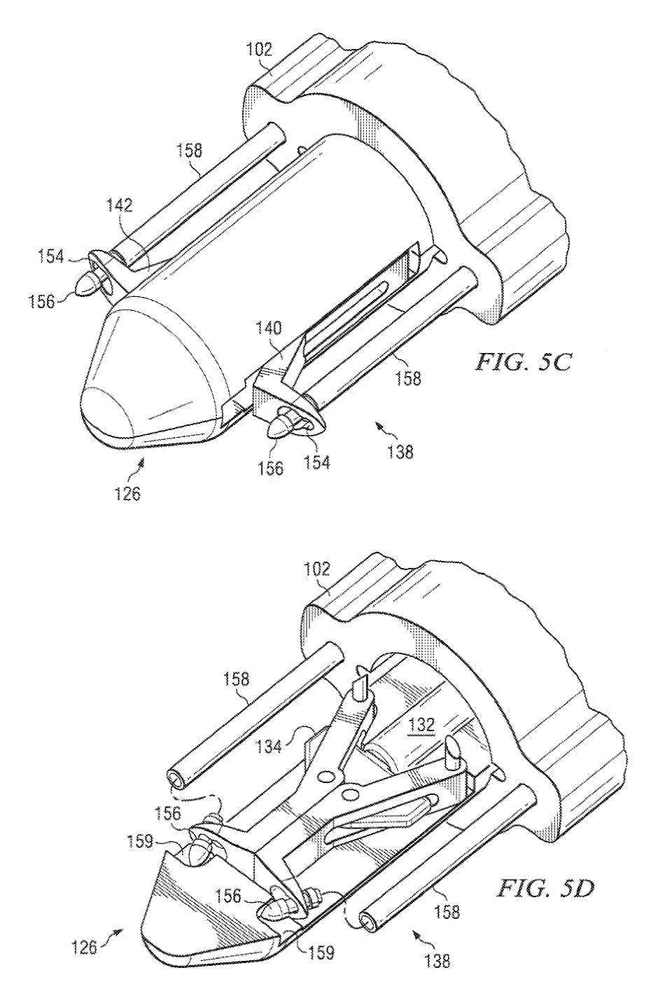

Referring now to FIGS. 5A-5D, the operation of the needle catcher mechanism 138 will be described. As shown in FIG. 5A, needle catchers 140, 142 are initially stowed within the inner tube 118. The drive rod 132 and actuator plate 134 are withdrawn and the hook portions 152 of needle catchers 140, 142 are disposed substantially within the deployment portion 130 of inner tube 118. As the surgeon presses on control knob 124, as described above, drive rod 132 extends actuator plate 134 causing drive pins 146 to travel outwards along drive slots 136, thereby pivoting needle catchers 140, 142 about pivot pins 144 and outward until the hook portions 152 are in line with the path of needle heads 156 as shown in FIG. 5B. As the surgeon presses actuator handle 114, needle pusher tubes 158 extend needle heads 156 into annular tissue and into receiving apertures 154 in needle catchers 140, 142, as shown in FIG. 5C. As the surgeon withdraws control knob 124, drive rod 132 and actuator plate 134 are withdrawn into their original positions, as shown in FIG. 5D, thereby retracting the needle catchers back to their un-deployed positions within inner tube 118, and drawing the needle heads 156 with sutures attached within inner tube 118 as well. The sutures can then be brought back through the central opening and tied off, effecting retention of the tip 126 of inner tube 118 in the annulus.

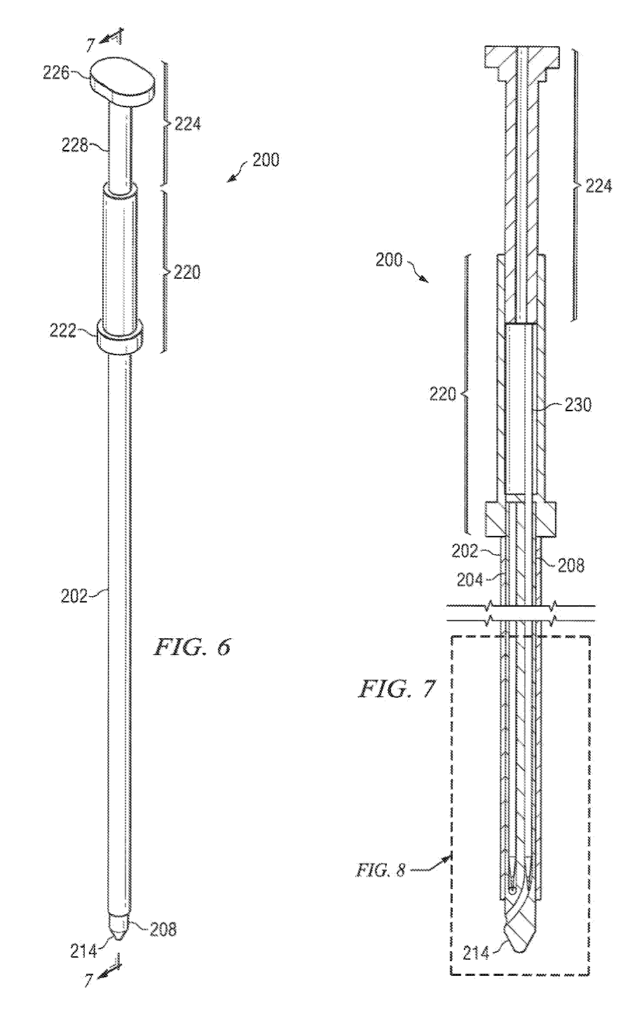

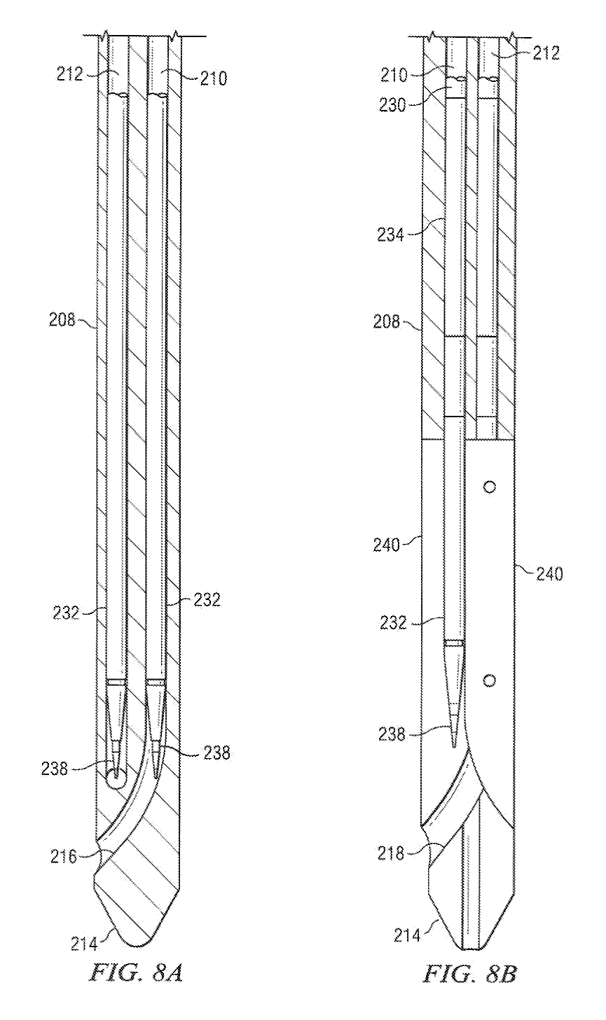

Referring now to FIGS. 6-7, an embodiment of an annular access device 200 provides minimally invasive access to the interior of an intervertebral disc, a stable working platform for treatment, and closure of the annulotomy. The annual access device 200 comprises an outer tube 202 having a central channel 204 running lengthwise therethrough, and an inner assembly disposed within the central channel 204. Referring also to FIGS. 8A-8B, the inner assembly comprises an inner tube 208 having at least two internal channels 210, 212 running lengthwise therethrough. The inner tube 208 extends beyond the distal end of outer tube 202. At a distal end of inner tube 208 is formed as a generally conical tip 214 having a rounded point so that inner tube 208 functions as a blunt obturator. Internal channels 210, 212 connect to exit ports 216, 218 formed in the inner tube 208 behind the tip 214.

An inner tube cap 220, having a collar portion 222, is coupled to a proximal end of the inner tube 208 at collar portion 222. A control knob 224 has a handle portion 226 and a shaft portion 228, and is coupled to inner tube cap 220 whereby the shaft portion 228 passes through a proximal end of the inner tube cap 220. The control knob 224 is further coupled to pusher tubes 230 disposed within internal channels 210, 212. Hollow, flexible anchors 232, preferably fabricated from a flexible polymer or other bio-resorbable plastic material, are disposed within internal channels 210, 212 at distal ends thereof. Flexible drive rods 234 are slidably disposed within internal channels 210, 212 between the anchors 232 and the pusher tubes 230. The drive rods 234 can be fabricated from a flexible metal alloy such as Nitinol, a nickel-titanium alloy. Sutures are attached to each anchor 232 and lie within pusher tubes 234. Needles 238 pass through the hollow anchors 232 and extend from the forward tip of the anchors to assist in penetration of the annulus and insertion of the anchors in the annulus. Filler plates 240 separate the channels 210, 212 as the channels cross over each other to connect to exit ports 216, 218.

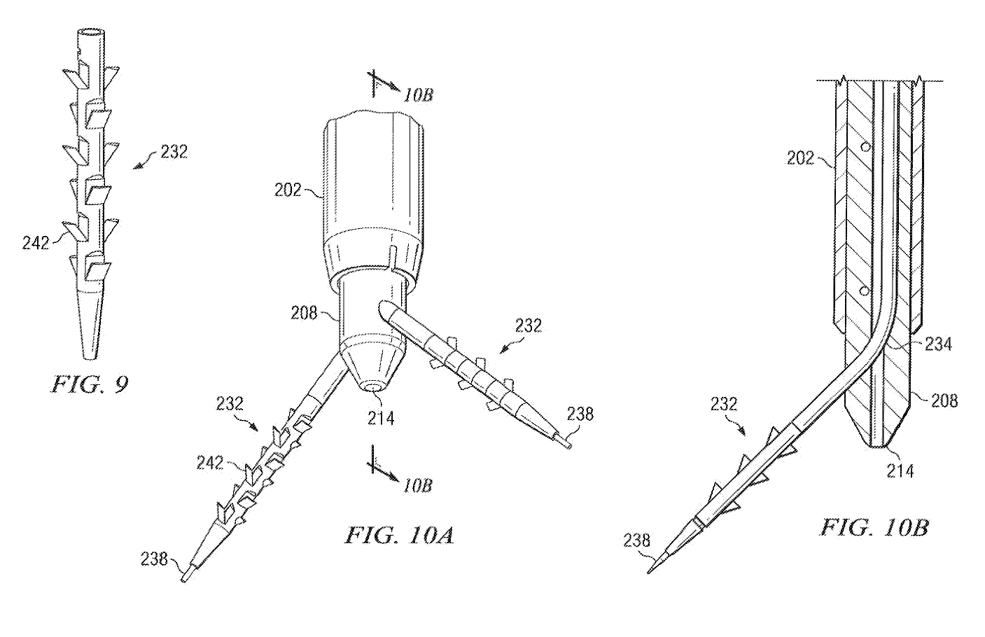

In operation, the surgeon presses knob 224 to actuate the annular access device and deploy the anchors 232. Referring to FIG. 9, each anchor 232 has a plurality of flexible barbs 242 that engage the annular tissue. Referring also to FIGS. 10A-10B, anchors 232 deploy from opposite sides of the obturator, inner tube 208. As the tip 214 will be in contact with annular tissue at this time, the anchors 232 are then driven into the annular tissue with the aid of needles 238. The needles can then be withdrawn, and the anchors 232 are held within the annular tissue so that sutures attached to the anchors can be drawn tight to hold the cannula, outer tube 202 of annular access device 200, in place. Thereafter, the sutures can be drawn tighter and tied-off to provide closure of the annulotomy.

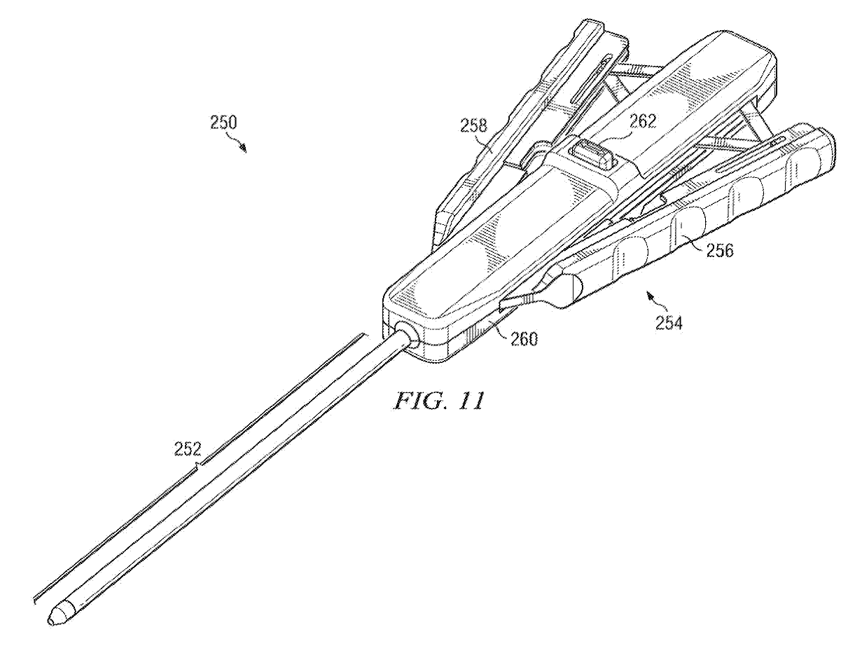

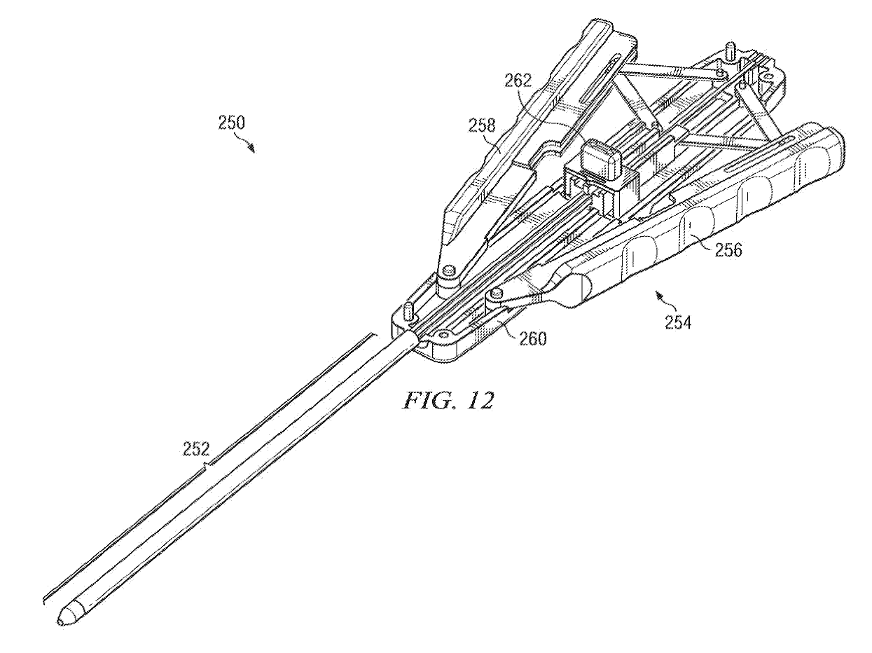

Referring now to FIGS. 11-15C, an embodiment of an annular access device 250 provides minimally invasive access to the interior of an intervertebral disc, a stable working platform for treatment, and closure of the annulotomy. The annular access device 250 comprises a shaft assembly 252 and a handle assembly 254. The shaft assembly 252 is similar to the cannula and obturator shown in FIG. 6 as outer tube 202 and inner tube 208, respectively, including flexible barbed anchors 232 carried within inner channels 210, 212. The handle assembly 254 comprises dual actuation levers 256, 258 pivotally mounted to a handle body 260, and a selector switch 262 that allows the surgeon to actuate the device 250 to deploy one anchor at a time or to deploy both anchors simultaneously.

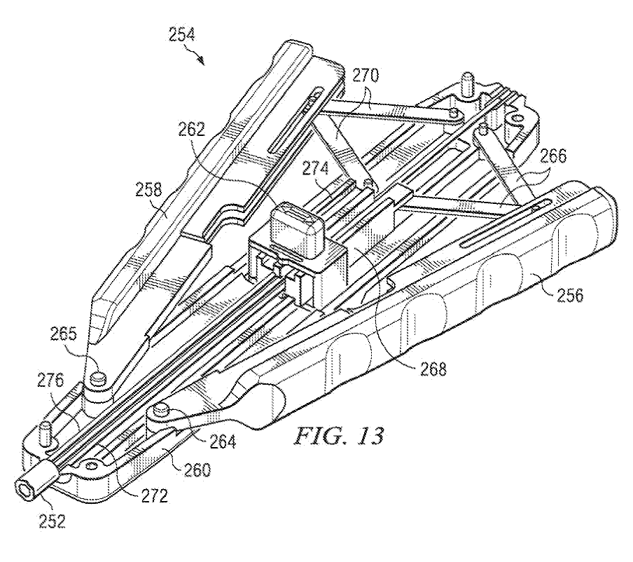

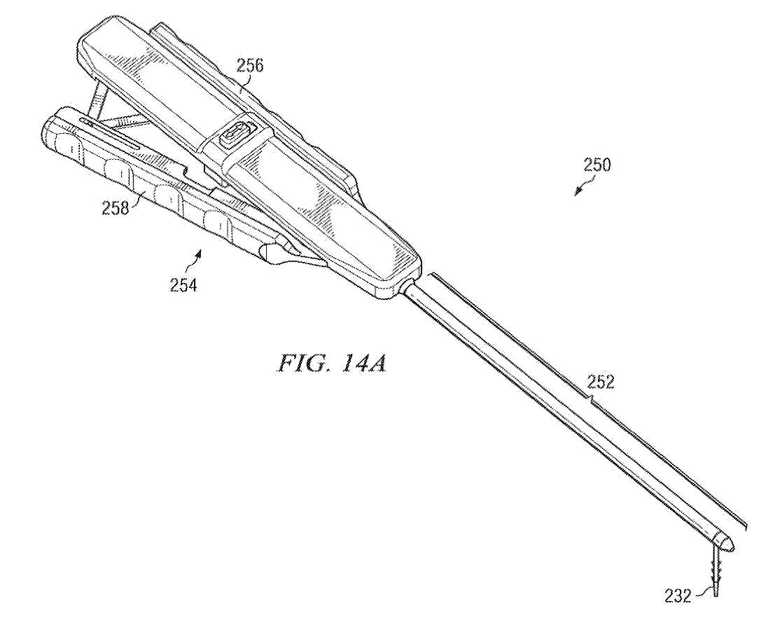

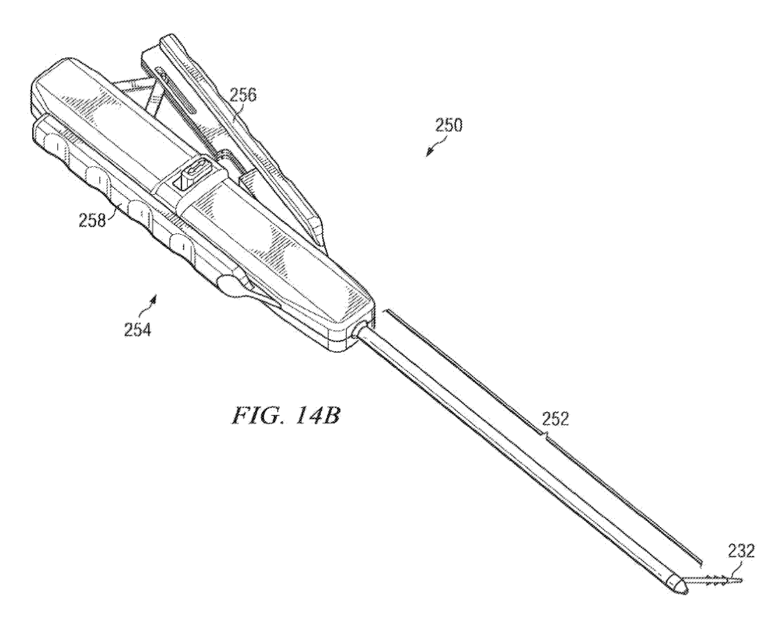

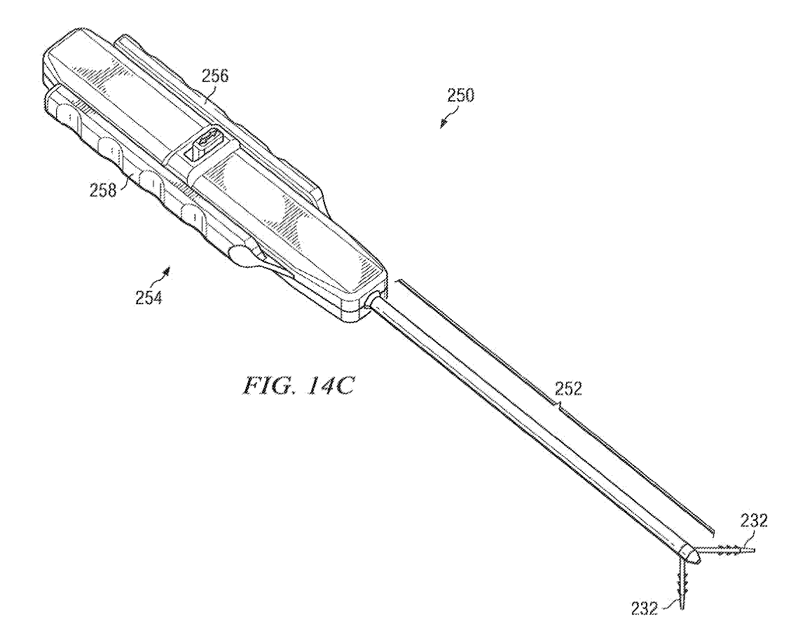

Referring now to FIG. 13, wherein one half of the handle body 260 is removed for clarity, actuation lever 256 is pivotally mounted at pivot pin 264 on handle body 260. Link members 266 operatively couple actuation lever 256 to a shuttle 268 slidably mounted within the handle body 260. The shuttle 268 engages a drive tube 272 that extends into the shaft assembly 252 to deploy one of the flexible barbed anchors, as shown in FIG. 14A. Likewise, actuation lever 258 is pivotally mounted on handle body 260, and link member 270 operatively couples actuation lever 258 to a shuttle 274 slidably mounted within the handle body 260. The shuttle 274 engages a drive tube 276 that extends into the shaft assembly 252 to deploy another of the flexible barbed anchors, as shown in FIG. 14B. Selector switch 262 can be operated from either side of handle assembly 254 to engage both shuttles 268, 274 and deploy both of the flexible barbed anchors simultaneously, as shown in FIG. 14C.

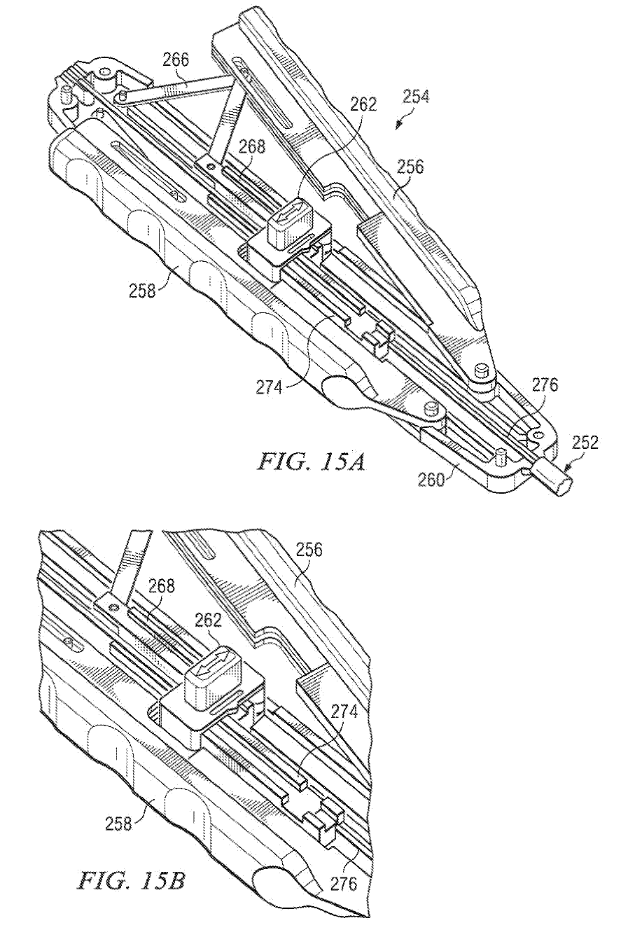

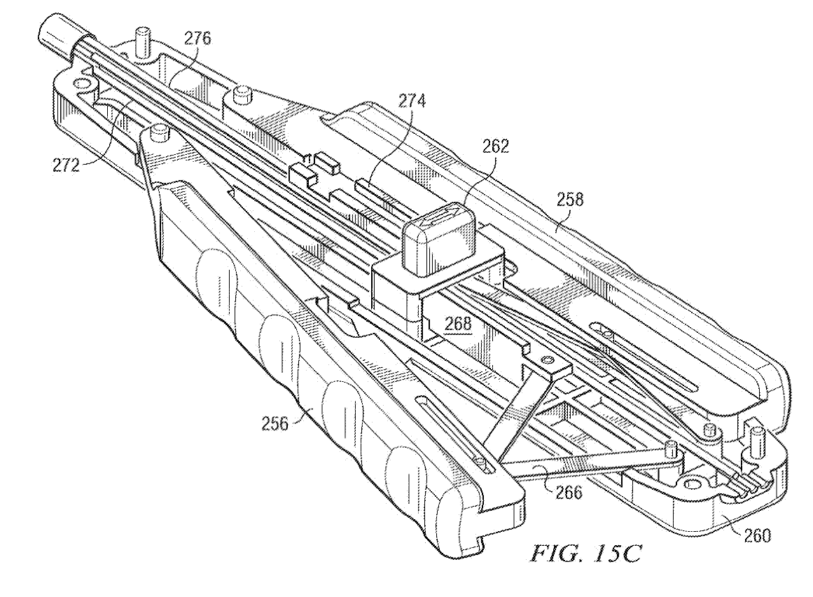

Referring now to FIGS. 15A-15C, wherein one half of the handle body 260 is removed for clarity, actuation lever 258 is pressed into the handle assembly 254, extending the linkage 270 and driving shuttle 274 forward. This action deploys a flexible anchor 232 as shown in FIG. 14A. Handle 256 remains extended from the handle body 260, link members 266 remain un-extended, and shuttle 268 remains in its original, rearward position.

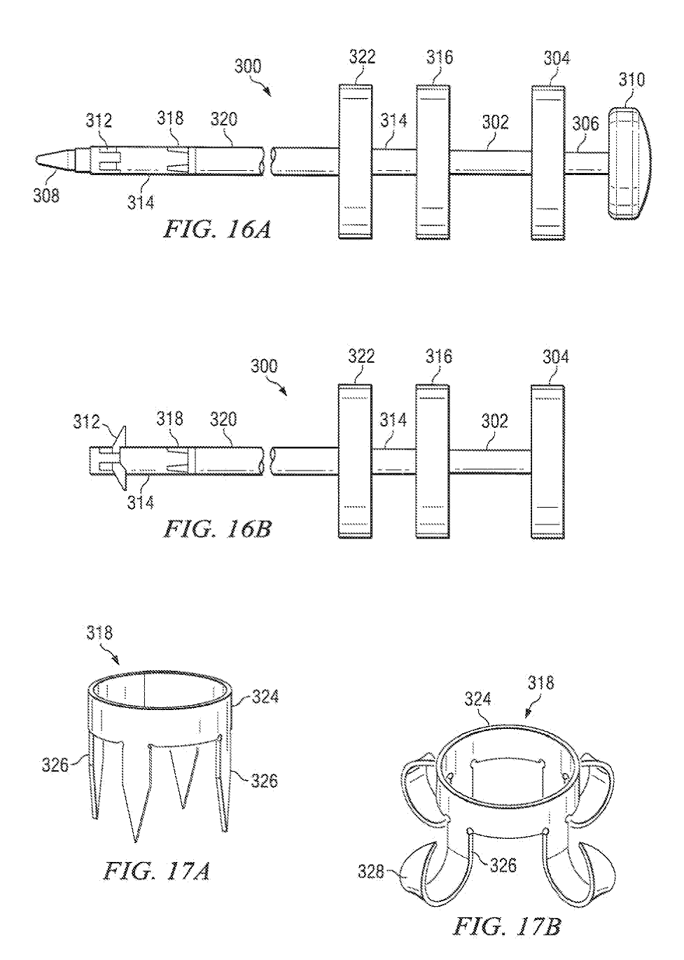

Referring now to FIGS. 16A-16B, an embodiment of an annular access device 300 provides minimally invasive access to the interior of an intervertebral disc, a stable working platform for treatment, and closure of the annulotomy. The annular access device 300 comprises a cannula 302 having a handle 304 at a proximal end thereof and an obturator 306 having a blunt tip 308 at a distal end and a handle 310 at a proximal end. The obturator 306 is initially disposed within the cannula 302. The device 300 further comprises a deployable anvil apparatus and a deployable staple apparatus. The deployable anvil apparatus comprises deployable anvils 312, an anvil actuation sleeve 314 slidably disposed over the cannula 302, and an anvil actuation handle 316 connected to a proximal end of the anvil actuation sleeve 314. The deployable staple apparatus comprises a staple 318, a staple actuation sleeve 320 slidably disposed over the anvil actuation sleeve 314, and a staple actuation handle 322 connected to a proximal end of the staple actuation sleeve 320.

For optimum performance, the cannula 302 has the following attributes: the cannula has an inner diameter of 5 mm or less to allow 5 mm maximum diameter surgical instruments to pass through; the cannula can be fixed in place so that position can be maintained during surgery; and the device can close the annulotomy by approximating tissue through the cannula. These objectives are accomplished by employing the anvil and staple apparatus. The anvils 312 act as means for stabilizing the cannula 302 in place and also act as the backing for the staple 318 to be deployed and directed through tissue. The staple 318 is used advantageously to close the annulotomy by approximating tissue through the cannula 302, as will be described hereinafter.

The device 300 as shown in FIG. 16A is delivered through the skin to the location of the desired surgical procedure. The outer diameter shows the anvils 312 and the staple 318 exposed, however, a polymer sheath may be advantageous to protect the staple and allow atraumatic movement of the device to the desired site. The anvil actuation sleeve 314 is advanced over the cannula distally to deploy the anvils 312 as shown in FIG. 16B. As the anvils 312 are deployed the device 300 is withdrawn slightly. This withdrawal allows the anvils 312 to seek the path of least resistance, which is the space between the annular tissue and the bone. When the anvils 312 are fully deployed, the cannula 302 is locked in place. The surgeon may perform the surgical procedure at this point. The anvils 312 may take several different shapes; they may extend straight out, or have curves to guide the staple to a desired shape as it is advanced through the tissue. Various manufacturing processes may be implemented to achieve desired shapes and strength of the anvils 312. Among the processes would be: coining, stamping, laser cutting, and heat treating

FIG. 17A depicts the staple 318 in the un-deployed state, comprising a circular band 324 and a plurality of legs 326 extending therefrom. This staple may take several configurations and be constructed from several different materials. Stainless Steel and titanium are two options of materials. The legs 326 of the staple may have multiple configurations as well: straight, tapered, curved, and there may be cutouts along the length of the leg to allow for a prescribed deformation as the staple 318 contacts the anvil 312 and moves through the tissue. The staple 318 may have 4 or more legs as well.

At the conclusion of the procedure, the staple 318 is deployed by advancing the outer sleeve 320 to force the staple 318 through the tissue and into the anvil 312. As the anvil 312 is contacted, the legs 326 of the staple 318 move outward and follow the path of the anvil 312 until the staple 318 is fully deployed in tissue. Preferably, the ends 328 of legs 326 of staple 318 curl back around, as shown in FIG. 17B, so that ideal tissue engagement is achieved and approximation of tissue can be accomplished.

The anvils 312 are retracted by pushing the anvils into the site and pulling the anvil actuation handle 316 to withdraw the anvil actuation sleeve 314 and pull the anvils 312 back along the diameter of the cannula 302. After the staple 318 is deployed and the anvils 312 are retracted, the ring 324 at the top of the staple 318 preferably remains exposed above the tissue. The ring 324 of the staple 318 may embody different configurations such that a crimping tool, when closing the ring, comes together in the effective manner to fully approximate tissue.

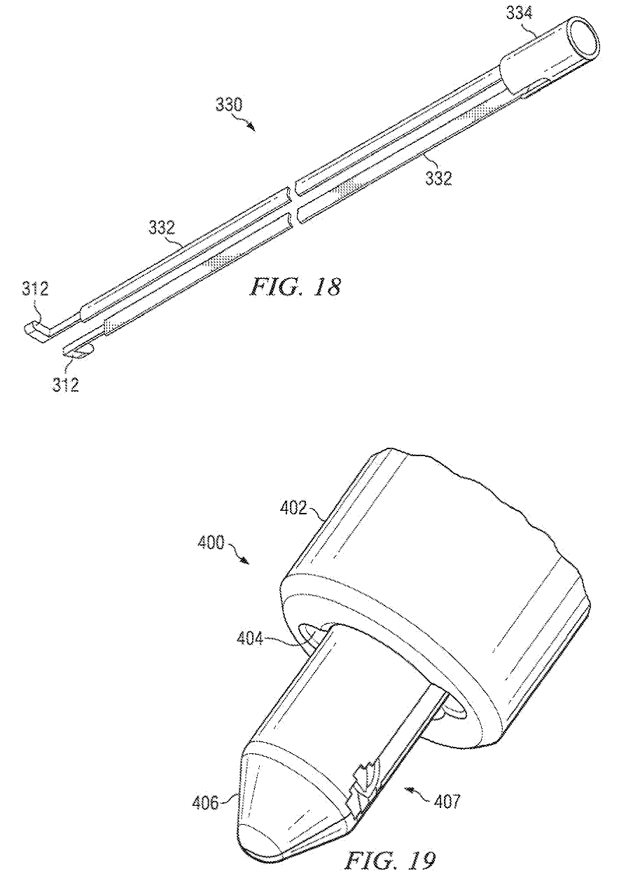

FIG. 18 depicts a representative anvil assembly 330 that can be used in the device 300. The anvil assembly comprises two anvils 312 attached to legs 332, which are in turn attached to a sleeve 334. The sleeve 334 is slidably disposed around a cannula and connected to an actuation handle, such as cannula 302 and actuation handle 316 as shown in FIG. 16A-16B. The anvils 312 are preferably resiliently connected to legs 332 to permit the anvils to be retracted initially and deployed later.

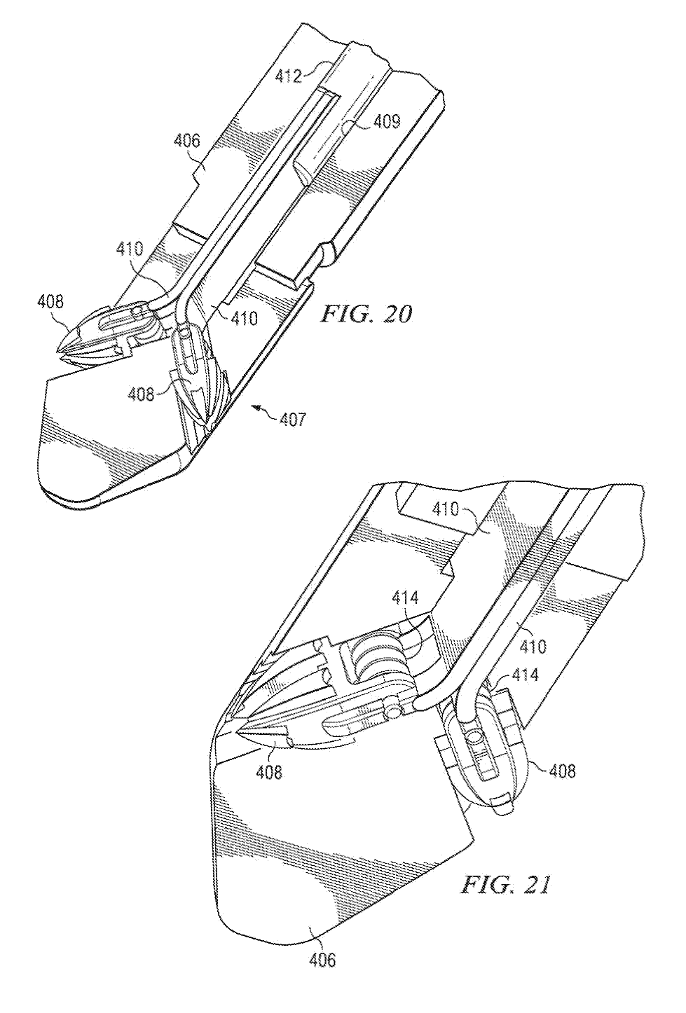

Referring now to FIGS. 19-24, an embodiment of an annular access device 400 provides minimally invasive access to the interior of an intervertebral disc, a stable working platform for treatment, and closure of the annulotomy. The annular access device 400 comprises a cannula 402 having a central channel 404, and a blunt obturator 406 having deployment slots 407 and a central working channel 409. Anchors 408 are initially stowed within deployment slots 407, as shown in FIG. 19-21, and are coupled to flexible drive bars 410. A drive rod 412 is slidably disposed within the central working channel 409 and is coupled at a distal end to flexible drive bars 410. The drive rod 412 is preferably coupled at a proximal end to an actuation mechanism (not shown) having a handle as hereinbefore described. Each anchor 408 has a pulley 414 about which sutures 416 are routed.

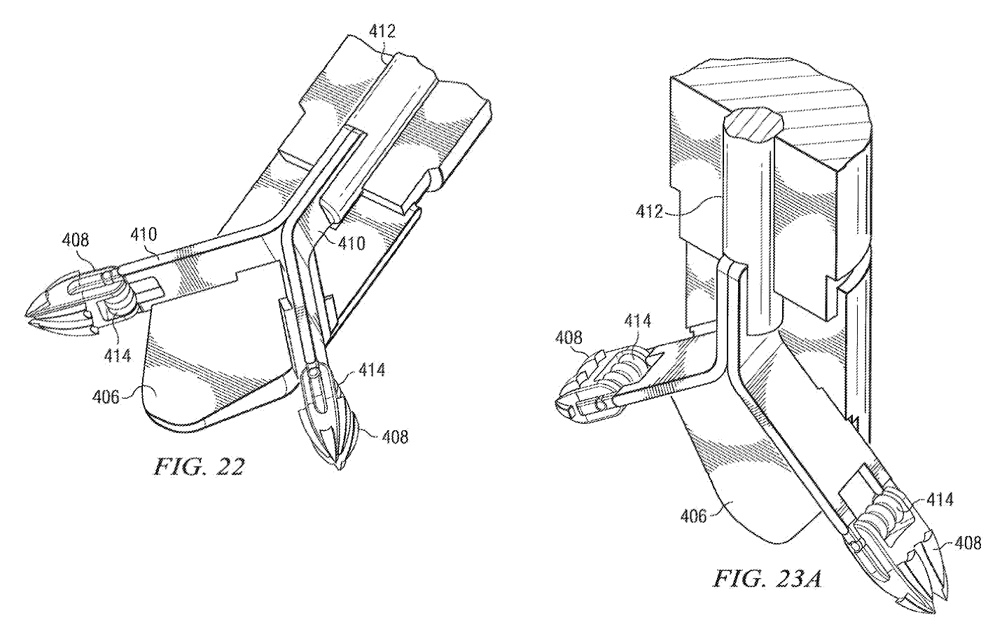

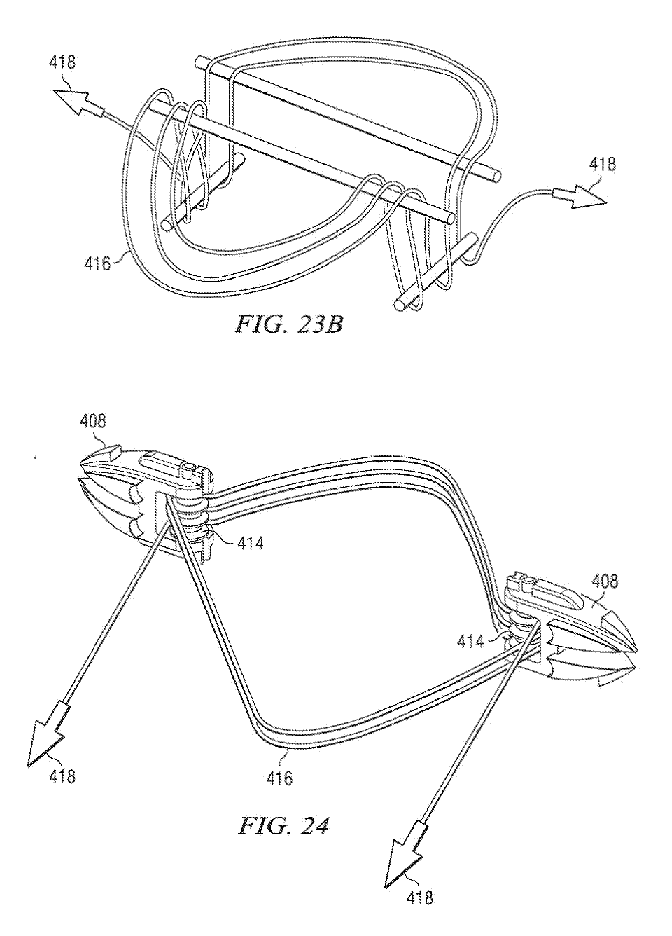

In operation, the device 400 is brought into contact with the site at which surgery is performed as hereinbefore described, wherein the distal end of the obturator 406 enters an incision initially made with a trephine, and the device is actuated by the surgeon to deploy the anchors 408 as shown in FIG. 22-24. The anchors enter tissue surrounding an annulotomy, the sutures are deployed as shown in FIG. 23B and FIG. 24, and the sutures are drawn tight to secure the device 400 to the surgical site. At the conclusion of the surgical procedure, the device 400 is withdrawn leaving the anchors 408 in place, and the sutures are drawn in the direction of arrows 418 for closure of the annulotomy with a net or web of sutures. The net or web of sutures effectively prevent extrusion of implanted material.

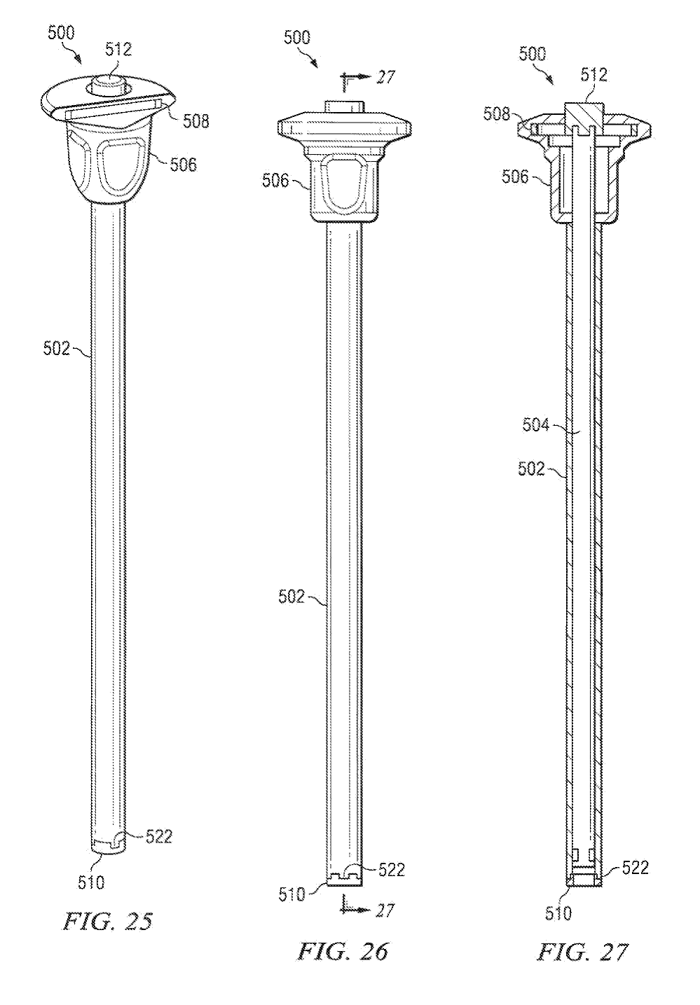

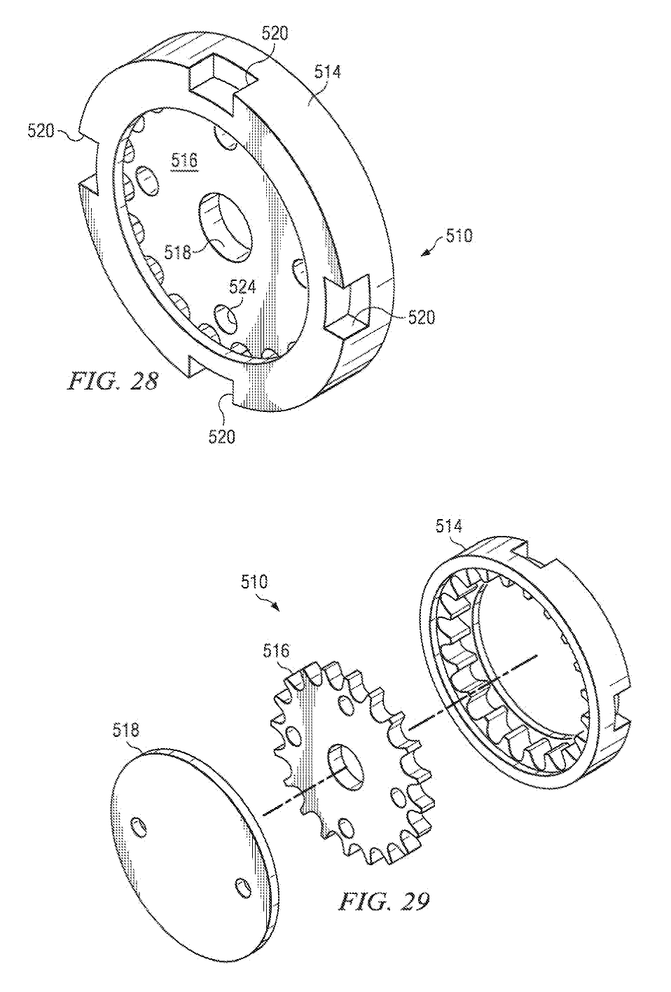

Referring Now to FIGS. 25-29, an embodiment of a suture locking device 500, comprises and outer tube 502, an inner tube 504 disposed within outer tube 502, and a handle 506 mounted on a proximal end of the device 500. Rotatably mounted within handle 506 is a knob 508 coupled to inner tube 504 via a release button 512. Rotation of the knob 508 causes concomitant rotation of the inner tube 504 and a suture locking mechanism 510 mounted to the distal end of inner tube 504. The release button 512 mounted in the top of handle 506 is coupled to knob 508 and engages the proximal end inner tube 504.

The suture locking mechanism 510 comprises a cap 514, an insert 516, and a bottom plate 518. The cap 514 has four holding slots 520 evenly spaced around the periphery thereof. Holding slots 520 mate with tabs 522 on the distal end of outer tube 502 for mounting the suture locking mechanism 510 to outer tube 502. The insert 516 is coupled to the rotatable inner tube 504. The bottom plate 518 is mounted in a releasable manner to a distal end of cap 514. Suture holes 524 are provided in the bottom plate for passing two ends of sutures therethrough.

In operation, the distal end of device 500 is positioned adjacent the site of a surgical procedure, such as an annulotomy as hereinbefore described. At the completion of a surgical procedure, the surgeon can rotate knob 508, thereby rotating the inner tube 504 and the insert 516. Sutures passing through suture holes 524 are wound together over the bottom plate 518 to secure the bottom plate to the annulotomy. The surgeon can then press the release button 512 to extend inner tube 504 and release the bottom plate 518 from the cap 514. The device 500 can then be removed from the surgical site.

It is understood that the present invention can take many forms and embodiments. Accordingly, several variations may be made in the foregoing without departing from the spirit or the scope of the invention.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered obvious and desirable by those skilled in the art based upon a review of the foregoing description of preferred embodiments. Accordingly, it is appropriate that the appended claims be construed broadly and in a manner consistent with the scope of the invention.

We claim:

1. An apparatus for performing a surgical operation on an intervertebral disc wherein the disc has an annulus on which an annulotomy may be performed, the apparatus comprising:
a cannula having a proximal end and a distal end;
an obturator coupled to the distal end of the cannula and being adapted for insertion into the annulotomy;
an annulus engaging member operatively coupled to the obturator and adapted to engage the annulus; and
an actuator operatively coupled to the member in such a manner that when the actuator is actuated the member engages the annulus thereby securing the apparatus to the annulus.

2. The apparatus of claim 1, wherein the annulus engaging member comprises a needle, and wherein the apparatus further comprises a needle catcher operatively coupled to the actuator in such a manner that when the actuator is actuated the needle catcher receives the needle following insertion of at least a portion of the needle through at least a portion of the annulus.

3. The apparatus of claim 1 wherein the annulus engaging member comprises an anchor.

4. The apparatus of claim 3 wherein the anchor comprises a barb.

5. The apparatus of claim 1 wherein the cannula has a longitudinal axis extending between the proximal and distal ends, and wherein the annulus engaging member is configured to extend from the cannula at an acute angle with respect to the longitudinal axis of the cannula.

6. The apparatus of claim 1 wherein the actuator further comprises a rotatable knob operatively coupled to the annulus engaging member in such a manner that when the knob is rotated the annulus engaging member engages the annulus.

7. The apparatus of claim 1 wherein the actuator further comprises a handle having a first position and a second position and being operatively coupled to the annulus engaging member in such a manner that when the handle is moved from the first position to the second position the annulus engaging member engages the annulus.

8. The apparatus of claim 1 wherein the annulus engaging member comprises a staple anvil.

9. The apparatus of claim 8 further comprising a staple and a staple actuator, the staple being operatively coupled to the staple actuator in such a manner that when the staple actuator is actuated the staple engages the staple anvil.

10. The apparatus of claim 1 wherein the cannula has an inner diameter of approximately 5 mm or less.

11. The apparatus of claim 1 wherein the annulus engaging member is configured to detach from the obturator.

12. The apparatus of claim 1 wherein the annulus engaging member is further adapted to engage at least one suture in such a manner that the suture is drawn through at least a portion of the annulus when the actuator is actuated.

13. The apparatus of claim 12 wherein the at least one suture further comprises a web of sutures.

14. An apparatus for performing a surgical operation on an intervertebral disc wherein the disc has an annulus on which an annulotomy may be performed, the apparatus comprising:
a cannula having a proximal end and a distal end;
an obturator coupled to the distal end of the cannula and being adapted for insertion into the annulotomy;
means for engaging the annulus operatively coupled to the obturator; and
means for actuating operatively coupled to the means for engaging in such a manner that when the means for actuating is actuated the means for engaging engages the annulus thereby securing the apparatus to the annulus.

15. A method of performing a surgical operation on an intervertebral disc wherein the disc has an annulus, the method comprising:
inserting an obturator into an annulotomy in the annulus, the obturator including a member adapted to engage the annulus and being coupled to a cannula; and
engaging the annulus with the member by actuating an actuator operatively coupled to the member thereby securing the obturator to the annulus.

16. The method of claim 15 wherein the engaging the annulus with the member further comprises drawing at least one suture through at least a portion of the annulus.

17. The method of claim 15 further comprising approximating a portion of the annulus with a staple.

18. The method of claim 15 further comprising leaving the member in the annulus.

19. A suture locking device for use in closing an annulotomy of an intervertebral disc wherein the disc has an annulus, the apparatus comprising:
a plate including at least two suture holes wherein each hole is adapted to receive a suture passing there through from the annulus;
an actuator operatively coupled to the plate in such a manner that the plate rotates when the actuator is actuated whereby the sutures are wound together generally adjacent the plate thereby securing the plate to the annulus; and
a release mechanism operatively coupled to the plate in such a manner that when the release mechanism is actuated the plate is released from the suture locking device.

20. The apparatus of claim 19 wherein the release mechanism further comprises a push button.

21. The apparatus of claim 19 wherein the actuator further comprises a rotatable knob and wherein rotating the knob actuates the actuator.

22. A system apparatus for performing a surgical operation on an intervertebral disc wherein the disc has an annulus on which an annulotomy may be performed, the system comprising:
an annular access device including:
a cannula having a proximal end and a distal end;
an obturator coupled to the distal end of the cannula and being adapted for insertion into the annulotomy;
an annulus engaging member operatively coupled to the obturator and adapted to engage the annulus; and
an actuator operatively coupled to the member in such a manner that when the actuator is actuated the member engages the annulus thereby securing the apparatus to the annulus; and
a suture locking device including:
a plate including at least two suture holes wherein each hole is adapted to receive a suture passing there through from the annulus;
an actuator operatively coupled to the plate in such a manner that the plate rotates when the actuator is actuated whereby the sutures are wound together generally adjacent the plate thereby securing the plate to the annulus; and
a release mechanism operatively coupled to the plate in such a manner that when the release mechanism is actuated the plate is released from the suture locking device.
